(19) United States
(12) Patent Application Publication
(10) Pub. No.: US 2014/0072531 A1
(43) Pub. Date: Mar. 13, 2014

(54) METHOD FOR PREPARING MICROPARTICLES WITH REDUCED INITIAL BURST AND MICROPARTICLES PREPARED THEREBY

(71) Applicant: SK CHEMICALS CO., LTD., Seongnam Si (KR)

(72) Inventors: Hong Kee KIM, Gunpo-si (KR); Kyu Ho Lee, Suwon-si (KR); Joon-Gyo OH, Suwon-si (KR); Bong-Yong Lee, Seoul (KR)

(73) Assignee: SK CHEMICALS CO., LTD., Seongnam Si (KR)

(21) Appl. No.: 14/085,170

(22) Filed: Nov. 20, 2013

Related U.S. Application Data
(63) Continuation of application No. PCT/KR2012/004000, filed on May 21, 2012.

(30) Foreign Application Priority Data
May 20, 2011 (KR) 10-2011-0048105

(51) Int. Cl. A61K 9/16; A61K 31/4196

(52) U.S. Cl. CPC A61K 9/1635; A61K 31/4196; A61K 9/1694
USPC 424/85.2; 514/383; 424/85.4; 424/85.1; 514/5.9; 514/11.7; 514/11.3; 514/9.8; 514/11.6; 514/9.7; 514/11.8; 514/7.7; 514/8.5; 514/9.6; 514/8.2; 514/8.9; 514/9.1; 514/8.1; 514/8.8; 514/11.9; 514/7.8; 264/5

(57) ABSTRACT

A method for preparing polymer microparticles with a reduced initial burst, and the polymer microparticles prepared thereby, the method including: contacting polymer microparticles with an alcohol aqueous solution, the polymer microparticles prepared thereby, and use for drug delivery of the polymer microparticles.

### CROSS REFERENCE TO RELATED APPLICATIONS

This application is a Continuation of the International Application No. PCT/KR2012/004000, filed on May 21, 2012, which claims priority from and the benefit of Korean Patent Application No. 10-2011-0048105, filed on May 20, 2011, which are hereby incorporated by reference for all purposes as if fully set forth herein.

### BACKGROUND

1. Field

Aspects of the present invention relate to a method for preparing polymer microparticles having a reduced initial burst, and the polymer microparticles prepared thereby. More particularly, aspects of the present invention relate to a method for preparing drug-loaded polymer microparticles with a reduced initial burst comprising contacting the polymer microparticles with an alcohol aqueous solution, the polymer microparticles prepared thereby, and use for drug delivery of the polymer microparticles.

2. Discussion of the Background

Conventional injectable formulations such as solution, suspension, and emulsion are quickly removed from the body after administration, and therefore frequent administration is essentially needed for treatment of chronic diseases. Microencapsulation has been developed to solve the problem, and referred to a production process for encapsulating drugs in microspheres (hereinafter, the term microsphere will include nanospheres) consisting of high molecular compounds. Microspheres are usually in a size of μm units, and can be administered to a human or animal by intramuscular or subcutaneous injection. Further, microspheres can be produced to have a variety of drug release rates, so that the period of drug delivery can be controlled. Therefore, even if a therapeutic drug is administered only once, its effective concentration can be maintained over a long period of time, and the total administration amount of therapeutic drug can be minimized to improve the drug compliance in patients. Accordingly, pharmaceutical companies are very interested in the production of polymeric microsphere loaded with drugs.

However, in a microparticle system of prolonged release formulation, in many cases, a high initial drug release, that is, an initial burst, occurs. This is because a drug is quickly diffused through water-filled pores existing in surfaces and/or the insides of microparticles, and water channels connecting them. This initial burst may cause side effects such as a toxic response. Thus, in development of the microparticle system, an initial burst should be minimized or eliminated.

For this reason, there have been many attempts to reduce an initial burst, such as coating of prepared microparticles with another material, insertion of microparticles into another controlled release system (hydrogel, etc.), adjustment of a formulation, adjustment of a preparation process, and removal of a drug on a microparticle surface through washing with a solvent. Each of these methods has a disadvantage/limitation as described below.

The coating of the microparticles or the insertion of the microparticles into another controlled release system (hydrogel, etc.) generally requires an additional material and/or an additional process. This reduces economic efficiency, and complicates a product development process. Further, there is a limitation that an additional material to be used in these methods is very limited, since the microparticles are used as injections.

In the case where an initial burst is reduced through adjustment of a formulation or a preparation process, the adjustment generally changes even the entire release profile. Thus, it is very difficult to obtain a profile with regular continuance for a required period of time, together with a reduction of an initial burst. Also, even if a required profile can be obtained through many attempts, the profile is available only in a specific drug or a specific polymer in the adjusted formulation or the adjusted preparation process. Thus, for a new drug or a new polymer, it is required to find and apply a completely novel method.

The method of removing a drug from a microparticle surface through washing with a solvent has been mainly used in with hydrophilic drugs. In O/W and W/O/W preparation methods, from among methods for preparing microparticles, a polymer is dissolved in an organic solvent, and then, is emulsified in a water-soluble solution, and hardened. Thus, in the case of a hydrophilic drug, since the hydrophilic drug has a tendency to be dispersed in an external water soluble phase, the drug frequently exists in a large amount on the surfaces of microparticles. This causes a high initial drug release. In the method, such a drug present in a large amount on the surface is washed with water and removed, so as to reduce an initial burst of a final formulation. The effect of the method, on hydrophilic drugs, has been reported. However, in the case of a hydrophobic drug, an organic solvent is needed to wash off the drug. The organic solvent is generally also used as a solvent in raw materials (PLGA(poly-lactic-co-glycolic acid), PLA(poly-lactic acid), etc.) of microparticles, and thus, causes a destruction of a microparticle structure.

### SUMMARY

Accordingly, aspects of the present invention relate to a method of reducing an initial burst, which is applicable to PLGA and PLA microparticles prepared by coacervation (phase separation), spray-drying, solvent evaporation/extraction, solvent ammonolysis (or hydrolysis), etc. Also, aspects of the present invention relate to a method of reducing an initial burst, which is applicable to hydrophobic drug-containing microparticles as well as hydrophilic drug-containing microparticles.

Accordingly, an object of the present invention is to provide a novel method of preparing polymer microparticles with a reduced initial drug release. Also, an object of the present invention is to provide a method of reducing an initial drug release of polymer microparticles prepared by various methods.

In order to accomplish this object, regarding the method for preparing drug-loaded polymer microparticles with a reduced initial burst comprising the steps of: (a) preparing drug-loaded polymer microparticles; and (b) contacting the drug-loaded polymer microparticles with an alcohol aqueous solution.

In order to accomplish another object of the present invention, the present invention provides drug-loaded polymer microparticles with a reduced initial burst, prepared by the inventive method.

In order to accomplish still another object of the present invention, the present invention provides a drug delivery composition comprising the drug-loaded polymer microparticles with a reduced initial burst, prepared by the inventive method as an active ingredient.

In order to accomplish still another object of the present invention, the present invention provides a drug delivery method comprising administering an effective amount of the drug-loaded polymer microparticles with a reduced initial burst, prepared by the inventive method to a subject in need thereof.

In order to accomplish still another object of the present invention, the present invention provides a use of the drug-loaded polymer microparticles with a reduced initial burst, prepared by the inventive method for preparing an agent for drug delivery.

### BRIEF DESCRIPTION OF THE DRAWINGS

The accompanying drawings, which are included to provide a further understanding of the invention and are incorporated in and constitute a part of this specification, illustrate embodiments of the invention, and together with the description serve to explain the principles of the invention.

FIG. 1 shows measurement results of an initial burst rate of microparticles prepared with a concentration of 15% of a polymer within a solvent and

FIG. 2 shows measurement results of an effect on an initial burst by the treatment with an ethanol mixture according to a change of a temperature condition.

### DETAILED DESCRIPTION OF THE ILLUSTRATED EMBODIMENTS

Unless otherwise defined, all technical and scientific terms used herein have the same meaning as commonly understood by one of ordinary skill in the art to which this invention belongs. The following literatures provide general definitions of various terms used in the specification of the present invention: Singleton et al., DICTIONARY OF MICROBIOLOGY AND MOLECULAR BIOLOTY (2ded.1994); THE CAMBRIDGE DICTIONARY OF SCIENCE AND TECHNOLOGY (Walkered., 1988); and Hale & Marham, THE HARPER COLLINS DICTIONARY OF BIOLOGY.

Hereinafter, the present invention will be described in more detail.

The inventive method for preparing drug-loaded polymer microparticles with a reduced initial burst comprising:

(a) preparing drug-loaded polymer microparticles; and

(b) contacting the drug-loaded polymer microparticles with an alcohol aqueous solution so as to decrease a Tg of the polymer to a lower glass transition temperature of TO.

The inventive method of preparing the drug-loaded polymer microparticles with a reduced initial burst comprises the step of preparing polymer microparticles, before the step of contacting with an alcohol aqueous solution. Herein, the preparation of the polymer microparticles may be carried out by a conventional method known in the art.

Preferably, i) a solvent evaporation/extraction method or a solvent ammonolysis (or hydrolysis) method through emulsion, ii) a method using spray-drying or iii) a method using phase separation may be used. More preferably, i) a method of preparing O/W (oil-in-water), O/O (oil-in-oil) or W/O/W (water-in-oil-in-water) emulsion comprising a polymer compound, a drug and a dispersion solvent, and aggregating it into microparticles, ii) a method of spraying an organic solvent comprising a polymer compound, and a drug in heated air, thereby solidifying polymers and then aggregating them into microparticles or iii) a method of comprising phase separation in an organic solvent comprising a polymer compound and a drug by addition of a non-solvent, and transferring it to an additional non-solvent, thereby solidifying polymers and then aggregating them into microparticles may be used to prepare the polymer microparticles.

More specifically, in the method of preparing polymer microparticles by preparing emulsion and aggregating it into polymer microparticles, first, O/W, O/O or W/O/W emulsion including a polymer compound, a drug, and a dispersion solvent is prepared.

The preparation of emulsion may be carried out by a conventional method known in the art. More specifically, O/W or O/O emulsion may be prepared by adding a dispersed phase including a polymer compound and a drug to a dispersion solvent. Meanwhile, W/O/W emulsion may be prepared by preparing W/O emulsion through emulsification of an aqueous solution having a drug dissolved therein in a solvent having a polymer compound dissolved therein, and adding the W/O emulsion to a dispersion solvent.

In such drug-containing polymer microparticles, the emulsion is aggregated into microparticles by a solvent evaporation method or a solvent extraction method, or is aggregated into microparticles by ammonolysis or hydrolysis. In the preparation of emulsion through ammonolysis or hydrolysis, a water-insoluble organic solvent is further included, in which the water-insoluble organic solvent is converted into a water-soluble solvent through ammonolysis or hydrolysis, by addition of ammonia (an ammonolysis process) or an acid or base (a hydrolysis process).

The solvent evaporation method, but not limited thereto, may comprise the methods, for example, disclosed in U.S. Pat. Nos. 6,471,996, 5,985,309, and 5,271,945. In the method, a drug is dispersed or dissolved in an organic solvent having a polymer compound dissolved therein, and emulsified in dispersion medium such as water so as to prepare O/W (oil-in-water) emulsion, and then the organic solvent in the emulsion is diffused in the dispersion medium and evaporated through an air/water interface so as to form drug-containing polymer microparticles.

The solvent extraction method comprises a conventional solvent extraction used in preparation of drug-containing polymer microparticles, such as a method of effectively extracting an organic solvent in emulsion drops by using a large amount of solubilizing solvent.

Furthermore, as a method of employing both a solvent evaporation method and a solvent extraction method, for example, methods disclosed in U.S. Pat. Nos. 4,389,840, 4,530,840, 6,544,559, 6,368,632, and 6,572,894, may be used.

In the aggregation through ammonolysis, for example, the method disclosed in Korean Patent No. 918092 may be used. In the method, ammonolysis is induced in O/W, W/O/W, or O/O emulsion including a water-insoluble organic solvent by addition of ammonia to the emulsion while the water-insoluble organic solvent is converted into a water-soluble solvent, thereby aggregating microparticles.

In the aggregation through hydrolysis, for example, the methods disclosed in Korean Patent Application Nos. 2009-109809 and 2010-70407 may be used. In the methods, hydrolysis (a kind of hydrolysis of ester) is induced in O/W, W/O/W or O/O emulsion including water-insoluble organic solvent by addition of a base (such as NaOH, LiOH, KOH) or an acid (such as HCl, H₂SO₄) solution to the emulsion while the water-insoluble organic solvent is converted into a water-soluble solvent, thereby aggregating microparticles.

In the method of preparing polymer microparticles through spray drying, a polymer compound is dissolved in a volatile organic solvent, and a drug is dissolved or dispersed in the polymer solution. When the solution (or dispersion) is sprayed in heated air, the solvent is momentarily evaporated and the polymer is solidified, thereby forming polymer microparticles.

In the method of preparing polymer microparticles through phase separation (coacervation), after a polymer compound is dissolved in an organic solvent, a drug is dissolved in the polymer solution, is dispersed, in state of solid powder, or is dissolved in water and dispersed in the organic solvent. The solution (or dispersion) is added with a non-solvent in driblets to induce phase separation in the solution. Then, the solution is transferred to an additional non-solvent, thereby solidifying polymers and forming polymer microparticles.

In other words, the inventive method of preparing drug-loaded polymer microparticles with a reduced initial burst is characterized in that it includes the steps of (a) dissolving a polymer and a drug in a solvent, and aggregating them into microparticles and (b) contacting the aggregated polymer microparticles with an alcohol aqueous solution, so as to lower a Tg of the polymer compound down to TgΔ.

There is no limitation in the polymer compound used in the preparation method of the present invention, as long as it is a polymer compound known in the art. Preferably, the polymer compound may be selected from the group consisting of polylactic acid, polylactide, polylactic-co-glycolic acid, polylactide-co-glycolide (PLGA), polyphosphazene, polyiminocarbonate, polyphosphoester, polyanhydride, polyorthoester, lactic acid-caprolactone copolymer, polycaprolactone, polyhydroxyvalerate, polyhydroxybutyrate, polyamino acid, lactic acid-amino acid copolymer, and a mixture thereof.

The drug used in the present invention may include all of hydrophilic drugs and hydrophobic drugs and it may be used without limitation if it is able to be encapsulated to polymeric microshperes. Examples of the drug comprise progesterone, haloperidol, thiothixene, olanzapine, clozapine, bromperidol, pimozide, risperidone, ziprasidone, diazepam, ethyl loflazepate, alprazolam, nemonapride, fluoxetine, sertraline, venlafaxine, donepezil, tacrine, galantamine, rivastigmine, selegiline, ropinirole, pergolide, trihexyphenidyl, bromocriptine, benztropine, colchicine, nordazepam, etizolam, bromazepam, clotiazepam, mexazolum, buspirone, goserelin acetate, somatotropin, leuprolide acetate, octreotide, cetrorelix, octreotide acetate, gonadotropin, fluconazole, itraconazole, mizoribine, cyclosporin, tacrolimus, naloxone, naltrexone, cladribine, chlorambucil, tretinoin, carmustine, anagrelide, doxorubicin, anastrozole, idarubicin, cisplatin, dactinomycin, docetaxel, paclitaxel, raltitrexed, epirubicin, letrozole, mefloquine, primaquine, oxybutynin, tolterodine, allylestrenol, lovostatin, simvastatin, provastatin, atorvastatin, alendronate, salcatonin, raloxifene, oxadrolone, conjugated estrogen, estradiol, estradiol valerate, estradiol benzoate, ethinyl estradiol, etonogestrel, levonorgestrel, tibolone, norethisterone and piroxicam. The drug may also be macro molecules of proteins or nucleic acids, such as interleukin, interferon, tumor necrosis factor, insulin, glucagon, growth hormone, gonadotropin, oxytocin, thyroid stimulating hormone, parathyroid hormone, calcitonin, colony stimulation factor, erythropoietin, thrombopoietion, insulin-like growth factor, epidermal growth factor, platelet-derived growth factor, transforming growth factor, fibroblast growth factor, vascular endothelial growth factor, bone morphogenetic protein.

A method for preparing drug-loaded polymer microparticles with a reduced initial burst of the present invention is characterized by comprising the step of contacting polymer microparticles formulated by the methods such as the solvent evaporation/extraction, solvent ammonolysis (or hydrolysis), spray drying, or phase separation (coavervation) with an alcohol aqueous solution.

In the inventive method for preparing polymer microparticles, an alcohol aqueous solution may be that of 60% (v/v) or less. Preferably, it may be an alcohol aqueous solution with a range of 0% to 60% (v/v) and more preferably it may be an alcohol aqueous solution with a range of 0% to 50% (v/v), 1% to 50% (v/v), further more preferably it may be an alcohol aqueous solution with a range of 5% to 50% (v/v) and most preferably it may be an alcohol aqueous solution with a range of 10% to 40% (v/v). The lower limit of the content of alcohol in an alcohol aqueous solution may be 0.001, 0.01, 0.1, 1, 2, 3, 4, 5, 6, 7, 8, 9, or 10%, for example. The upper limit may be 60, 59, 58, 57, 56, 55, 54, 53, 52, 51, 50, 49, 48, 47, 46, 45, 44, 43, 42, 41, 40, 39, 38, 37, 36, 35, 34, 33, 32, 31, or 30%, for example.

Alcohols used herein may be a low carbon alcohol of C1 to C6 such as methanol, ethanol, propanol, isopropanol, butanol, pentanol and hexanol. Preferably, the alcohol may be ethanol.

In the alcohol aqueous solution used in the treatment, alcohol is preferably included in a range of 60% (vol %) or less in a case where many surface pores exist by a W/O/W method, or is in a range of less than 40% (vol %) in a case where a small amount of surface pores exist due to an O/W method. When alcohol is included in an amount of the above mentioned range, within the alcohol aqueous solution, the spherical shape of polymer microparticles may not be maintained. Instead, they may be aggregated as a whole. Further, when alcohol is in an amount of the above mentioned range, the initial drug release effect may not be sufficiently achieved.

By such treatment of alcohol, the Tg (glass transition temperature) of the polymer within the polymer microparticles is decreased to a reduced glass transition temperature (TgΔ). Thus, the surface/interior pore structures of polymer microparticles are closed or filled, thereby achieving an effect densifying the polymer microparticles. Herein, the TgΔ may be equal to, lower, or higher than the reaction temperature at which treatment with alcohol is carried out. When the TgΔ is lower than the reaction temperature or a predetermined temperature (for example, any temperature ranging from greater than 0 to 5° C., for example 1, 2, 3, 4 or 5° C., the heating step, as described below, may be not necessary. Also, when the TgΔ is equal to or higher than the reaction temperature, the heating step, as described below, is preferably added. Through such an effect, the structures allowing the drug within the polymer microparticles to be released to the outside are decreased. This seems to cause an effect of reducing an initial burst. In Comparative Example 2 of the present invention, such an effect was confirmed based on that alcohol treatment reduced the particle size of polymer microparticles.

The method of preparing drug-loaded polymer microparticles with a reduced initial burst may further comprise the step of contacting the drug-loaded polymer microparticles with an alcohol aqueous solution having a temperature higher than the TgΔ of the polymer.

In other words, the method may comprise the steps of: (a) dissolving a polymer and a drug in a solvent, and aggregating the polymer and drug into microparticles; (b) heating the polymer microparticles at a temperature higher than TgΔ of the polymer; and (c) contacting the microparticles with an alcohol aqueous solution, so as to lower Tg of the polymer to TgΔ.

Also, the heating step may be carried out after the contacting with an alcohol aqueous solution.

In other words, the method may comprise the steps of: (a) dissolving a polymer and a drug in a solvent, and aggregating the polymer and drug into microparticles; (b) contacting the microparticles with an alcohol aqueous solution, so as to lower Tg of the polymer to TgΔ; and (c) heating the microparticles to a temperature higher than TgΔ of the polymer.

The temperature of the heating may range from a temperature higher than TgΔtemperature by 4° C. to a temperature higher than TgΔ temperature by 50° C. In other words, it may range from TgΔ+4° C. to TgΔ+50° C. For example, it may range from TgΔ+4° C. to TgΔ+40° C.

When the temperature is less than TgΔ+4° C., the effect may not be achieved, and when the temperature is higher than TgΔ+50° C., the microparticles may be deformed.

After the temperature is raised up to a temperature higher than TgΔ, the microparticles are contacted with the alcohol aqueous solution, to reduce an initial drug release of the microparticles. The process may be ended without the heating step. Otherwise, the heating step and the treating step may be sequentially carried out. Further, for example, additional steps, such as a washing step and a drying step, may be further included before, during, or after each of the above mentioned steps.

In the preparation method of the present invention, the treatment of the polymer microparticles may be carried out within a predetermined temperature range for a predetermined time, by bringing the polymer microparticles into contact with the alcohol aqueous solution. For example, the treatment may be carried out by placing or immersing the microparticles in the alcohol aqueous solution.

The contacting time may be varied according to the kind of the polymer compound, the concentration of the alcohol aqueous solution, the contacting temperature, etc. For example, the contacting may be carried out for greater than 0 seconds to 48 hours or less.

Further, the case where the initial drug release is not reduced or is not sufficiently reduced by only the contacting with the alcohol aqueous solution is caused by the Tg of the polymer compound not being reduced to the reaction temperature or less (that is, TgΔ>reaction temperature). Thus, in the treatment step after heating, the temperature may be raised to a temperature higher than TgΔ temperature of the polymer compound, followed by an additional treatment step for greater than 0 seconds to 48 hours or less.

Tg represents a glass transition temperature at which molecules starts to move with an activity in a polymer compound. In general, a low molecular weight material in a solid phase is phase-transitioned from a solid phase to a liquid phase, by heating. On the other hand, a polymer in a solid phase is placed in a flexible state, not in a liquid phase, by heating, due to a change of a physical property thereof. The temperature causing such a change is referred to as Tg. According to the kind and combination of a polymer compound in use, each Tg value may be varied. For example, PLGA and PLA polymers, frequently used in preparation of polymer microparticles, have a Tg of about 50° C., which is noted in Table 1 below, based on the data of a manufacturer (Lakeshore).

TABLE 1

| Polymer | Tg (° C.) |
|---|---|
| Polyglycolide (PGA) | 35-40 |
| Poly(L-lactide) (LPLA) | 60-65 |
| Poly(DL-lactide) (DLPLA) | 45-55 |
| 8515 Poly(DL-lactide-co-glycolide) (DLPLG) | 45-52 |
| 75/25 DLPLA | 45-52 |
| 65/35 DLPLG | 45-50 |
| 50/50 DLPLG | 30-45 |

Tg, in accordance with the kind or the content of each polymer compound, may be confirmed by measurement according to manufacturer information, or DSC or TGA methods (Macromol. Res., Vol. 19, No. 11, (2011); C. G. Park et al., AAPS PharmSciTech, Vol. 9, No. 4, December (2008); Dorati et al.).

Further, the treatment time may be 48 hours or less, and preferably 24 hours or less. When the treatment time is excessively long, the drug within the microparticles may be dispersed into the ethanol aqueous solution. This may reduce the content of the drug within the microparticles. The lower limit of the treatment time may be 0.001, 0.01, 0.1, 1, 2, 3, 4, 5, 6, 7, 8, 9, 10 seconds, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 35, 40, 45, 50, 55 minutes, or 1 hour, for example. The upper limit may be 48, 47, 46, 45, 44, 43, 42, 41, 40, 39, 38, 37, 36, 35, 34, 33, 32, 31, 30, 29, 28, 27, 26, 25, or 24 hours, for example.

The present invention provides polymer microparticles prepared by the inventive method of preparing the polymer microparticles. The polymer microparticles of the present invention have a significantly reduced initial drug release, and thus, can significantly reduce side effects caused by the initial drug release.

Also, when the polymer microparticles of the present invention are used, it is possible to effectively deliver the drug included in the polymer microparticles. Thus, the present invention provides a drug delivery composition comprising the polymer microparticles as an active ingredient, the polymer microparticles being prepared by the preparation method of the present invention. The drug delivery composition of the present invention may comprise the polymer microparticles prepared by the preparation method of the present invention an amount of 1 to 99% (w/w), and in a carrier in an amount of 99% to 1% (w/w).

An agent comprised in the drug delivery composition of the present invention may be varied in accordance with diseases, as would be understood by skilled person in the art.

For the references, techniques for producing nucleotides and proteins mentioned herein are well described in Maniatis et al., Molecular Cloning: A Laboratory Manual, Cold Spring Harbor Laboratory, Cold Spring Harbor, N.Y. (1982); Sambrook et al., Molecular Cloning: A Laboratory Manual, 2d Ed., Cold Spring Harbor Laboratory Press(1989); Deutscher, M., Guide to Protein Purification Methods Enzymology, vol. 182. Academic Press. Inc., San Diego, Calif. (1990).

In Comparative Example, unlike microparticles prepared by a W/O/W method, when microparticles prepared by an O/W method were subjected to a treatment in an alcohol aqueous solution, the microparticles were aggregated, deformed, and lumped together or failed to show an initial burst reducing effect.

Accordingly, in order to solve the problem of aggregation in microparticles, in one example of the present invention, the content of ethanol was varied in the treatment. As a result, when an ethanol ratio was 40% or more, the microparticles were aggregated and lumped together after the treatment, while when an ethanol ratio was less than 40%, the microparticles were not aggregated, and their original spherical particle structures were maintained. Further, TgΔ according to the treatment of ethanol was confirmed.

In another Comparative Example, within a range where no aggregation occurs, the effect of a change of an ethanol concentration on an initial burst was examined. As a result, by the treatment at an ethanol/water ratio of 2:8, the initial drug release was not reduced, and by the treatment at 3:7, the initial drug release was reduced to some extent. However, the degree of the reduction did not result in a significant enough effect to fully serve the purpose.

Accordingly, in order to solve the problem that the degree of the reduction of the initial burst did not fully serve the purpose, another example of the present invention was carried out. Herein, the insufficient reduction of the initial burst was assumed to be caused by the Tg reduction effect through treatment with an ethanol aqueous solution not being sufficient. Thus, in order to solve the insufficiency of the Tg reduction, it was determined that, in the inverse way, when the reaction temperature itself is increased up to Tg (TgΔ) or more, decreased by the treatment with the ethanol aqueous solution, the release reduction effect can be achieved. Then, when the treatment of an ethanol-water mixture at 40° C. was additionally carried out, it was found that the initial burst can be reduced by about 40% to 65%, as compared to a non-treated case.

In another example of the present invention, in order to confirm the release reduction effect by treatment at a temperature of TgΔ or more of the polymer in use, the treatment with a mixture at an ethanol to water ratio of 2:8 was carried out at a treatment temperature of TgΔ+5° C. or TgΔ+40° C., for 30 minutes. As a result, it was found that, at each treatment temperature, the initial burst can be reduced.

In another example of the present invention, the effect according to a concentration of an ethanol: water mixture was examined. As a result, it was found that within a range of 10% to 50%, the initial burst can be reduced.

In another example of the present invention, on polymer microparticles in various states prepared by varying a particle size, a treatment time, a concentration of a polymer compound, it was determined if the treatment with an ethanol aqueous solution can be a general method for achieving an initial burst reducing effect. As a result, it was found that all of the polymer microparticles showed a high initial burst reducing effect.

In another example of the present invention, it was determined if a change in the temperature during the treatment with an ethanol aqueous solution has an effect on an initial burst. As a result, it was found that in the case of a composition that cannot show an initial drug release reducing effect by only the treatment at a temperature of lower than Tg, it is impossible to achieve an initial drug release reducing effect by the treatment only at a temperature of higher than TgΔ. Further, it was found that the initial burst rate reducing effect can be achieved only when the treatment was carried out at room temperature lower than Tg and then at a temperature of 40° C. higher than TgΔ.

In other words, one polymer composition, in which Tg is lowered to a reaction temperature or less (room temperature in Example) by only the treatment with an ethanol aqueous solution due to low Tg of the polymer composition (that is, TgΔ<reaction temperature), for example, a composition having a polymer with a low molecular weight, can show an initial drug release reducing effect only by treatment at a reaction temperature. However, the other polymer composition, in which Tg is not lowered to a reaction temperature or less (room temperature in Example) by only the treatment with an ethanol aqueous solution due to high Tg of the polymer composition (that is, TgΔ>reaction temperature), for example, a composition having a polymer with a high molecular weight, can show an initial drug release reducing effect through addition of a treatment process at a raised reaction temperature.

Accordingly, the present invention provides a novel method of preparing polymer microparticles with a reduced initial drug release. The method of the present invention may be useful for preparing a new formulation of drug that can prevent various side effects caused by excessive release of an agent.

Hereinafter, the present invention will be described in detail with reference to the following Examples and Comparative Examples. However, examples below are for illustrative purpose only and are not constructed to limit the scope of the present invention.

### Comparative Example 1

Preparation of microparticles according to a conventional method, and measurement of an initial burst rate.

<1-1> Preparation of Polymer Microparticles and Treatment with Ethanol at a High Concentration.

An ethyl formate solution containing 15% (w/v) PLA 2E and a 0.5 wt % polyvinyl alcohol (PVA) aqueous solution were mixed, and stirred so as to prepare O/W emulsion. The prepared emulsion was reacted with a NaOH solution, and distilled water (DW) was then added thereto. Through dispersion and filtration, microparticles were collected. The collected microparticles were re-dispersed in a 0.1 wt % polyvinyl alcohol (PVA) aqueous solution, and then filtered. Then, microparticles were collected and dried (this method in this paragraph is designated as F072 hereinafter.

The prepared microparticles with a size of 83.6 μm were placed in 67 ml of mixture liquid at an ethanol:water ratio=5:5 (v/v), and sufficiently mixed. As a result, within 1 minute, the microparticles were aggregated, deformed, and lumped together. Then, an initial burst rate cannot be measured.

<1-2> Preparation of Polymer Microparticles, and Treatment at a Low Temperature

An ethyl formate solution containing 10% (w/v) PLA 4.5E and a 0.5 wt % polyvinyl alcohol (PVA) aqueous solution were mixed, and stirred so as to prepare O/W emulsion. The prepared emulsion was reacted with a NaOH solution, and added with distilled water (DW). Through dispersion and filtration, microparticles were collected. The collected microparticles were re-dispersed in a 0.1 wt % polyvinyl alcohol (PVA) aqueous solution, and then filtered. Then, microparticles were collected and dried (F104-1).

The prepared microparticles with a size of 80.4 μm were treated with a solution having a mixture at an ethanol to water ratio of 2:8(v/v), at room temperature (a temperature lower than TgΔ), for 60 minutes. Then, through filtration, the microparticles were collected, and the collected microparticles were dried in a freeze-dryer (F104-2).

The microparticles were collected and the initial burst of a drug was measured. As a result, it was found that the initial burst was not reduced as noted in Table 2 below.

The measurement of an initial burst was carried out as follows: microspheres were placed in a dialysis membrane, immersed in a 37° CPBS (phosphated buffered saline), and then released by continuous shaking at 100 rpm. After a predetermined time (6 hours or 24 hours), the amount of a released drug was measured by UPLC (Ultra performance liquid chromatography).

TABLE 2

| | Initial Drug Release % (24 hr) |
|---|---|
| No treatment of ethanol:water mixture | 9.34 ± 3.55% |
| Treatment of ethanol:water mixture with a | 10.53 ± 0.59% |
| ratio of 2:8 | |

### Example 1

Preparation of the Inventive Microparticles According to an Ethanol Concentration.

<1-1> Preparation of Polymer Microparticles According to an Ethanol Concentration.

In order to examine if the aggregation of microparticles is affected by an ethanol concentration, the following experiment was carried out by varying the ethanol concentration.

Microparticles with a size of 83.6 μm, prepared by PLA 2E polymer (preparation condition: F072), and microparticles with a size of 80.4 μm, prepared by PLA 4.5E polymer (preparation condition: F104-1) were treated with 67 ml of a mixture, at an ethanol to water ratio of 1:9, 2:8, 3:7, 4:6, and 5:5. As a result, both kinds of microparticles were aggregated and lumped together when the ethanol ratio was 40% or more, in other words, when treated with the mixture at an ethanol to water ratio of 4:6, and 5:5. On the other hand, when an ethanol ratio was less than 40%, the microparticles were not aggregated even after 60 minutes of the treatment, and their original spherical particle structures were well maintained.

In a separate experiment, in the case of microparticles prepared by a W/O/W method, the microparticles were not aggregated even at an ethanol ratio of 50%, while as described above, in the case of microparticles prepared by an O/W method, the microparticles were aggregated at an ethanol ratio of 40% or more.

The cause of this difference was understood as follows. In the microparticles prepared by a W/O/W method, a large amount of surface pores exist while in the microparticles prepared by an O/W method, no surface pores exist. According to existence/nonexistence of surface pores, the amount of water molecules included in the microparticle surfaces is varied. This difference in water molecules was assumed to make a difference in Tg, thereby making a difference in the degree of softening of a polymer. Thus, it was understood that such a difference makes a difference in the degree of aggregation of particles.

Accordingly, since the microparticles prepared by an O/W method showed a different physical property from the microparticles prepared by a W/O/W method, it an effective specific treatment condition needed to be determined.

<1-2> Preparation of Polymer Microparticles, and TgΔ According to Treatment With Ethanol.

An ethyl formate solution containing 15% (w/v) of PLA 2E, PLA 4.5E, or PLGA 7525 7E, and a 0.5 wt % polyvinyl alcohol (PVA) aqueous solution were mixed, and stirred so as to prepare O/W emulsion. The prepared emulsion was reacted with a NaOH solution, and added with distilled water (DW). Through dispersion and filtration, microparticles were collected. The collected microparticles were re-dispersed in a 0.1 wt % polyvinyl alcohol (PVA) aqueous solution and then filtered (use of PLA 2E: designated as P4, use of PLA 4.5E: designated as P5, use of PLGA 7525 7E: designated as P6).

The prepared microparticles were placed in 50 ml of mixture liquid at an ethanol is to water ratio of 1:9, 2:8, 4:6 or 5:5 and sufficiently mixed. Through filtration, the microparticles, in a wet state, were collected, and their TgΔ was measured by DSC. The result is noted in Table 3 below.

TABLE 3

| | TgΔ (° C.) | | |
|---|---|---|---|
| Conc. of | P4 | P5 | P6 |
| EtOH (%) | PLA 2E | PLA 4.5E | PLGA 7525 7E |
| 0 | 39.0 | 43.8 | 41.1 |
| 10 | 28.3 | — | — |
| 20 | 23.8 | 33.5 | — |
| 40 | — | — | 29.07 |
| 50 | — | — | 27.73 |

### Comparative Example 2

Measurement of an Initial Burst Rate of Microparticles Prepared by an O/W Method.

An ethyl formate solution containing anastrozole (hydrophilic drug) and 10% (w/v) PLA 4.5E, and a 0.5 wt % polyvinyl alcohol (PVA) aqueous solution were mixed, and stirred so as to prepare O/W emulsion. The prepared emulsion was reacted with a NaOH solution, and added with distilled water (DW). Through dispersion and filtration, microparticles were collected. The collected microparticles were re-dispersed in a 0.1 wt % polyvinyl alcohol (PVA) aqueous solution, and then filtered. Then, microparticles were collected and dried (F104-5).

The prepared microparticles were treated with a solution having a mixture at an ethanol to water ratio of 2:8 and 3:7 (=ethanol mixture), at room temperature, for 60 minutes. Then, through filtration, the microparticles were collected, and dried (F104-6 and F104-8, respectively). The microparticles as prepared above, containing anastrozole (hydrophilic drug), with a size of 81.2 μm, were collected, and their initial burst rate was measured.

As a result, as noted in Table 4 below, the treatment of the mixture at an ethanol to water ratio of 2:8 did not reduce an initial drug release, and the treatment of the mixture at a ratio of 3:7 reduced the release to some extent, although it was statistically insignificant. Thus, although it is expected to reduce the initial burst by increasing an ethanol ratio, the initial burst rate reducing effect at a ratio of 3:7 was not sufficient. Further, when the ethanol ratio was increased, at the ethanol to water ratio of 4:6, the particles were already aggregated. Accordingly, it was found that only the treatment of an ethanol aqueous solution at room temperature is not effective in microparticles having a large amount of surface pores, prepared by a W/O/W method, or the like, and also is not effective in microparticles having few surface pores, prepared by an O/W method. Thus, it was found that an improvement is required.

TABLE 4

| | Particle Size | Initial Drug |
|---|---|---|
| | (μm) | Release % (24 hr) |
| No treatment of ethanol:water mixture | 81.2 | 13.43 ± 0.40% |
| Treatment of ethanol:water mixture with | 91.8 | 12.77 ± 1.32% |
| a ratio of 2:8 | | |
| Treatment of ethanol:water mixture with | 68.2 | 10.14 ± 2.61% |
| a ratio of 3:7 | | |

In view of the particle size, the particle size at the 3:7 treatment resulting in a release reducing effect was smaller than that at the 2:8 treatment, causing no release reducing effect. This is because Tg of PLA was reduced by the ethanol-water mixture, thereby softening PLA. Thus, pores and channels within the microparticles were destroyed while densifying the microparticles and reducing the size of the microparticles.

### Example 2

Preparation of Microparticles According to an Additional Temperature Condition of Tg or More, and Measurement of an Initial Burst Rate.

In the case where Tg of a polymer in use was relatively low, when Tg was lowered by the treatment of an ethanol-water mixture down to room temperature or less (experimental condition), an initial burst reducing effect can be achieved. On the other hand, in the case where Tg of a polymer in use was relatively high, an effect of reducing Tg of PLA down to room temperature or less (experimental condition) was not sufficient by the treatment at an ethanol to water ratio of 2:8, in this experiment.

Accordingly, in order to solve the insufficiency of the Tg reduction, it was determined that, when the reaction temperature is raised to TgΔ or more, the release reduction effect can be achieved. Thus, in preparation of microparticles, the treatment of an ethanol-water mixture at 40° C. was additionally carried out. Then, the initial burst rate was measured.

For this, in the same manner as described in Comparative Example 2 (F104-6, F104-8), the microparticles were prepared, except that the treatment of an ethanol-water mixture at 40° C. was additionally carried out. In other words, after the treatment with the mixture at an ethanol to water ratio of 2:8 for 60 minutes, the treatment temperature of the same mixture was raised to 40° C., and the treatment was further carried out for 60 minutes (F104-7). Also, the treatment with the mixture at an ethanol to water ratio of 3:7 was carried out in the same manner (F104-9).

As a result, as noted in Table 5 below, it was found that when the treatment temperature of the ethanol-water mixture was raised up to TgΔ or more of a polymer, the initial burst can be reduced by about 51% to 65%.

TABLE 5

| | Initial Drug |
|---|---|
| | Release % (24 hr) |
| No treatment of ethanol:water mixture | 13.43 ± 0.40 |
| (FF100104-5) | |
| Treatment of ethanol:water mixture with a ratio of | 6.61 ± 0.18 |
| 2:8 at room temp. and leave at 40° C. (FF100104-7) | |
| Treatment of ethanol:water mixture with a ratio of | 4.65 ± 0.18 |
| 3:7 at room temp. and leave at 40° C. (FF100104-9) | |

### Example 3

Preparation of Microparticles at a Temperature of TgΔ or More, and Measurement of an Initial Burst Rate.

In order to determine the release reducing effect at a temperature of TgΔ or more of a polymer in use, the microparticles were prepared in the same manner as described in Example 1-2 (P4), except that the treatment temperature of a mixture at an ethanol to water ratio of 2:8 was 28° C. (TgΔ+5° C., and 63° C. (TgΔ+40° C., and the treatment time was 30 minutes (treatment at 28° C. P4-1, treatment at 63° C. P4-2). After the treatment at respective temperatures, it was examined if the initial burst was reduced.

As a result, as noted in Table 6 below, it was found that when the treatment temperature of an ethanol-water mixture was raised up to TgΔ or more, the initial burst can be reduced.

TABLE 6

| | Initial Drug |
|---|---|
| | Release % (24 hr) |
| No treatment of ethanol:water mixture (P4) | 19.89 ± 1.97 |
| Treatment of ethanol:water mixture with a ratio of 2:8 | 15.31 ± 1.23 |
| at 28° C. (TgΔ + 5° C.) (P4-1) | |
| Treatment of ethanol:water mixture with a ratio of 2:8 | 14.94 ± 0.15 |
| at 63° C. (TgΔ + 40° C.) (P4-2) | |

### Example 4

Preparation of Microparticles According to an Ethanol Mixture Concentration, and Measurement of an Initial Burst Rate.

According to the concentration of an ethanol-water mixture, the degree of an initial burst may be varied. Microparticles were prepared in the same manner as described in Example 1-2 (P4, P6), except that the ethanol treatment concentration was changed (P4-1:ethanol concentration 10%, P4-2:ethanol concentration 20%, P6-1:ethanol concentration 40%, P6-2:ethanol concentration 50%). Then, on the microparticles prepared according to respective conditions, it was determined if the initial burst was reduced.

As a result, as noted in Table 7 below, it was found that when the microparticles were treated with the solution having an ethanol-water mixture at a concentration of 10%, 20%, 40%, and 50%, the initial burst can be reduced.

TABLE 7

| | Initial Drug |
|---|---|
| | Release % |
| | (24 h) |
| No treatment of ethanol:water mixture (P4) | 19.89 ± 1.97 |
| Treatment of ethanol:water mixture with a ratio of 1:9 at | 17.22 ± 1.69 |
| 33° C. (TgΔ + 5° C.) (P4-1) | |
| Treatment of ethanol:water mixture with a ratio of 2:8 at | 15.31 ± 1.23 |
| 28° C. (TgΔ + 5° C.) (P4-2) | |
| No treatment of ethanol:water mixture (P6) | 3.23 ± 0.09 |
| Treatment of ethanol:water mixture with a ratio of 4:6 at | 1.28 ± 0.14 |
| 33° C. (TgΔ + 4° C.) (P6-1) | |
| Treatment of ethanol:water mixture with a ratio of | 0.79 ± 0.03 |
| ethanol:water 5:5 at 33° C.(TgΔ + 5° C.) (P6-2) | |

### Example 5

Preparation of Microparticles According to a Particle Size, and Measurement of an Initial Burst Rate.

The degree of an initial burst may be varied according to a microparticle size. Thus, microparticles with different particle sizes were prepared in the same manner as described in Example 2, except that the agitation rate was changed (F104-5:550 rpm, F105-1: 1000 rpm).

The microparticles prepared according to respective conditions were treated with a mixture at an ethanol to water ratio of 2:8 at room temperature and then at 40° C. for 60 minutes. Then, it was determined if the initial burst was reduced.

As a result, as noted in Table 8 below, it was found that the initial burst rate reducing effect of about 40 to 50% was obtained irrespective of a particle size.

TABLE 8

| | Particle Size | Initial Drug Release |
|---|---|---|
| | (μm) | % |
| Large particles (F104-5) | 81.2 | 24 hr: 13.43 ± 0.40 |
| Treatment of ethanol mixture to large | | 24 hr: 6.61 ± 0.18 |
| particles (F104-7) | | |
| Small particles (F105-1) | 53.6 | 6 hr: 6.03 ± 0.22 |
| Treatment of ethanol mixture to small | | 6 hr: 3.64 ± 0.08 |
| particles (F105-2) | | |

(Herein, F105-2 is performed in the same manner as F104-7 (agitation rate 550 rpm) except that the agitation rate is 1000 rpm)

### Example 6

Preparation of Microparticles According to a Treatment Time, and Measurement of an Initial Burst Rate.

In order to examine if an initial burst rate of microparticles is affected by a treatment time at a reaction temperature of higher than Tg, microparticles were prepared in the same manner as described in Example 5 (F105-1), except that the treatment time at 40° C. was 20 minutes or 60 minutes. Then, the initial burst rate was measured.

As a result, as noted in Table 9 below, in both cases where the treatment of the ethanol-water mixture at 40° C. after the treatment at room temperature was carried out for 20 minutes and 60 minutes, the initial burst reduction effect was obtained.

TABLE 9

| | Initial Drug Release % |
|---|---|
| | (6 hr) |
| No treatment of ethanol:water mixture | 6.03 ± 0.22 |
| (F105-1) | |
| Treatment of ethanol:water mixture with | 2.58 ± 0.07 |
| a ratio of 2:8 at room temp. at 40° C. for 20 mins | |
| (F105-8) | |
| Treatment of ethanol:water mixture with | 3.64 ± 0.08 |
| a ratio of 2:8 at room temp. at 40° C. for 60 mins | |
| (F105-2) | |

### Example 7

Measurement of an Initial Burst Rate of Microparticles According to a Change in Formulation.

In order to examine if the initial burst rate reducing effect by the treatment of an ethanol aqueous solution is affected by a change in formulation such as a change in the concentration of a polymer, microparticles were prepared in accordance with the composition/treatment condition noted in Table 10 in the same manner as described in Example 2, except that the concentration of a polymer within an organic solvent was 15% (w/v), and an additive is varied. Then, the initial burst rate was measured (F105-5, F105-3, and F105-6). The detailed condition of each method is noted in Table 10 below.

TABLE 10

| Condition for | | Treatment of |
|---|---|---|
| Prep. | Contents | Ethanol Mixture |
| F105-5 | anastrozole, PLA | X |
| F105-3 | anastrozole, PLA | ◯ |
| | | (at 40° C. for 60 mins |
| | | after room temp.) |
| F105-6 | anastrozole, PLA, D-mannitol | ◯ |
| | | (at 40° C. for 60 mins |
| | | after roon temp.) |

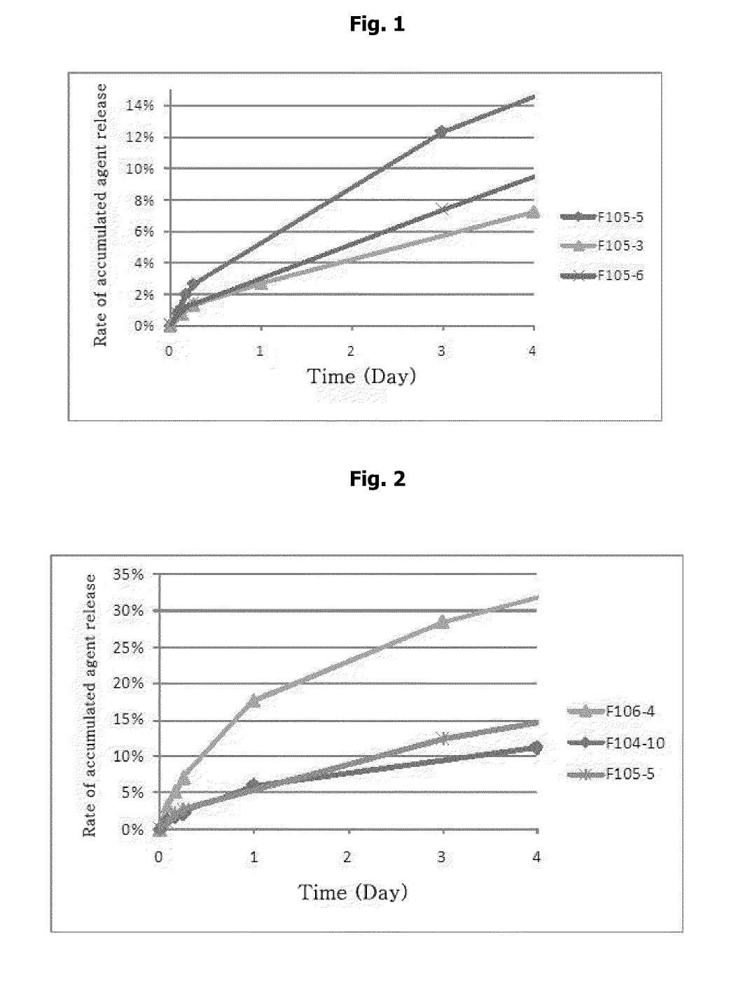

As a result, as shown in FIG. 1, it was found that even when the formulation was changed, the initial drug release reducing effect can be achieved by the treatment of the ethanol mixture.

### Example 8

Preparation of Microparticles According to a Temperature Condition, and Measurement of an Initial Burst Rate.

In order to examine if a change in the temperature condition in the treatment of an ethanol mixture has an effect on an initial burst, microparticles were prepared in accordance with the composition/treatment condition noted in Table 11 in the same manner as described in Example 7. Then, the initial burst rate was measured.

TABLE 11

| | | Treatment of | Treatment |
|---|---|---|---|
| Condition | | Ethanol Mixture at | of Ethanol |
| for Prep. | Contents | Room Temp. | Mixture at 40° C. |
| F105-5 | anastrozole, PLA | X | X |
| F104-10 | anastrozole, PLA | ◯ | X |
| F106-4 | anastrozole, PLA | X | ◯ |

As a result, as shown in FIG. 2, when the treatment of an ethanol mixture was carried out at any one temperature of room temperature or 40° C. the initial drug release reducing effect cannot be achieved. Accordingly, in the case of non-porous microparticles prepared by a polymer having high Tg in accordance with an O/W method, only the treatment of an ethanol aqueous solution at room temperature cannot reduce the initial burst rate. Further, only the treatment cannot reduce the initial burst rate at a raised temperature of 40° C. or more. Thus, it was found that the initial burst rate reducing effect can be achieved only when the treatment was carried out at a temperature of lower than Tg and then at a temperature of TgΔ or more.

What is claimed is:

1. A method for preparing drug-loaded polymer microparticles, comprising:
preparing drug-loaded polymer microparticles; and
contacting the drug-loaded polymer microparticles with an alcohol aqueous solution, so as to decrease a glass transition temperature (Tg) of the polymer to a glass transition temperature of TgΔ.

2. The method of claim 1, wherein the preparing of the drug-loaded polymer microparticles comprises:
preparing an O/W (oil-in-water), O/O (oil-in-oil), or W/O/W (water-in-oil-in-water) emulsion comprising a polymer, a drug, and a dispersion solvent; and
aggregating the emulsion to form the microparticles.

3. The method of claim 1, wherein the preparing of the drug-loaded polymer microparticles comprises:
dissolving a polymer and a drug in a solvent;
spraying the solvent into heated air; and
solidifying the polymer and the drug, thereby aggregating the polymer and drug into the microparticles.

4. The method of claim 1, wherein the drug-loaded polymer microparticles are prepared by:
adding a non-solvent to an organic solvent comprising a polymer and a drug, thereby inducing phase separation in a mixture;
transferring the mixture having separated phase to an additional non-solvent; and
solidifying the polymer and the drug by aggregation into the microparticles.

5. The method of claim 1, wherein the concentration of the alcohol in the alcohol aqueous solution is less than 60% (v/v).

6. The method of claim 5, wherein the concentration of the alcohol in the alcohol aqueous solution is with a range of 1% to 50% (v/v).

7. The method of claim 1, wherein the method further comprises contacting the drug-loaded polymer microparticles with an alcohol aqueous solution having a temperature higher than the TgΔ of the polymer.

8. The method of claim 7, wherein the concentration of the alcohol in the alcohol aqueous solution is less than 60% (v/v).

9. The method of claim 8, wherein the concentration of the alcohol in the alcohol aqueous solution is with a range of 1% to 50% (v/v).

10. The method of claim 7, wherein the temperature of the alcohol aqueous solution is in a range of TgΔ+4° C. to TgΔ+50° C.

11. The method of claim 1, wherein the polymer is selected from the group consisting of: a polylactic acid; a polylactide; a polylactic-co-glycolic acid; a polylactide-co-glycolide (PLGA); a polyphosphazene; a polyiminocarbonate; a polyphosphoester; a polyanhydride; a polyorthoester; a lactic acid-caprolactone copolymer; a polycaprolactone; a polyhydroxyvalerate; a polyhydroxybutyrate; a polyamino acid; a lactic acid-amino acid copolymer; and mixtures thereof.

12. The method of claim 1, wherein the drug is selected from the group consisting of: progesterone; haloperidol; thiothixene; olanzapine; clozapine; bromperidol; pimozide; risperidone; ziprasidone; diazepam; ethyl loflazepate; alprazolam; nemonapride; fluoxetine; sertraline; venlafaxine; donepezil; tacrine; galantamine; rivastigmine; selegiline; ropinirole; pergolide; trihexyphenidyl; bromocriptine; benztropine; colchicine; nordazepam; etizolam; bromazepam; clotiazepam; mexazolum; buspirone; goserelin acetate; somatotropin; leuprolide acetate; octreotide; cetrorelix; octreotide acetate; gonadotropin; fluconazole; itraconazole; mizoribine; cyclosporin; tacrolimus; naloxone; naltrexone; cladribine; chlorambucil; tretinoin; carmusitne; anagrelide; doxorubicin; anastrozole; idarubicin; cisplatin; dactinomycin; docetaxel; paclitaxel; raltitrexed; epirubicin; letrozole; mefloquine; primaquine; oxybutynin; tolterodine; allylestrenol; lovostatin; simvastatin; provastatin; atrovastatin; alendronate; salcatonin; raloxifene; oxadrolone; conjugated estrogen; estradiol; estradiol valerate; estradiol benzoate; ethinyl estradiol; etonogestrel; levonorgestrel; tibolone; norethisterone; and piroxicam.

13. A drug-loaded polymer microparticles prepared by the method of claim 1.

14. A drug delivery composition comprising the drug-loaded polymer microparticles of claim 13 as an active ingredient.

15. A drug delivery method comprising administering an effective amount of the drug-loaded polymer microparticles of claim 13 to a subject in need thereof.

16. An agent for drug delivery comprising the drug-loaded polymer microparticles of claim 13.

17. The method of claim 1, wherein the drug comprises macro molecules of proteins or nucleic acids selected from the group consisting of: interleukin; interferon; tumor necrosis factor; insulin; glucagon; a growth hormone; gonadotropin; oxytocin; thyroid stimulating hormone; parathyroid hormone; calcitonin; colony stimulation factor; erythropoietin; thrombopoietion; insulin-like growth factor; epidermal growth factor; platelet-derived growth factor; transforming growth factor; fibroblast growth factor; vascular endothelial growth factor; and bone morphogenetic protein.
